(19) United States
(12) Patent Application Publication
(10) Pub. No.: US 2007/0293428 A1
(43) Pub. Date: Dec. 20, 2007

(54) Methods of treating joint pain in a subject by using an anti-angiogenic agent

(76) Inventors: John M. Zanella, Cordova, TN (US); Sean M. Haddock, Memphis, TN (US)

Correspondence Address: FOX ROTHSCHILD, LLP, 997 LENOX DRIVE, LAWRENCEVILLE, NJ, 08648, US

(21) Appl. No.: 11/455,335

(22) Filed: Jun. 19, 2006

(51) Int. Cl. A61K 38/22; A61K 38/17; A61K 31/485; A61K 31/454; A61K 31/353

(52) U.S. Cl. 514/12; 514/323; 514/282; 514/454; 514/419; 514/561; 514/566

(57) ABSTRACT

A method for treating a joint pain in a subject comprising providing an effective amount of an anti-angiogenic agent to a synovial capsule adjacent to the joint.

### FIELD OF THE INVENTION

The present invention relates to methods of treating a joint pain in a subject by eliminating or reducing pain comprising providing an effective amount of an anti-angiogenic agent to a synovial capsule adjacent to the joint.

### BACKGROUND OF THE INVENTION

A normal joint where two bones meet is surrounded by a synovial capsule that protects and supports it. Cartilage covers and cushions the ends of two bones. The synovial capsule is lined with a type of tissue called the synovium, which produces a synovial fluid. The synovial fluid lubricates and nourishes the cartilage and bones inside the synovial capsule. In rheumatoid arthritis, the immune system attacks cells within the synovial joint and blood cells travel to the synovium and causes inflammation. This inflammation termed synovitis results in swelling and pain that are typical of rheumatoid arthritis. As rheumatoid arthritis progresses cartilage and bones within the joint are destroyed weakening the surrounding muscles, tendons, and ligaments that support and stabilize the joint. All these effects lead to pain often suffered by rheumatoid arthritis sufferers.

Subjects suffering from chronic rheumatoid arthritis pain incur emotional as well substantial detriment to productivity, disability and compensation. It is not uncommon for chronic arthritis sufferers to undergo drastic, highly invasive surgery that is both expensive and problematic to alleviate pain. Such surgery could range from recovery times ranging from six months to over a year, and in some cases patients experience subsequent pain levels to a degree that is equal to, or even exceeds, their pre-surgery levels.

In the prior art, angiogenesis and inflammation were believed to affect disease progression and pain in osteoarthritis. Inflammation can stimulate angiogenesis and angiogenesis was believed to facilitate inflammation. Inflammation sensitizes nerves, leading to increased pain. Innervation can also accompany vascularization of the articular cartilage causing pain even after inflammation has subsided. Inhibition of inflammation and angiogenesis may provide effective therapeutics for the treatment of osteoarthritis by improving symptoms and retarding joint damage. See C. S. Bonnet et.al., Rheumatology, Oxford Journals, 2005; 44: 7-16.

Vascularization is recognized to be key event in the development of normal cartilage and bone. By promoting the delivery of nutrients, oxygen and cells, blood vessels help maintain the structural and functional integrity of joints and soft tissue and may facilitate tissue repair and healing. The identification of pro-angiogenic mediators such as vascular endothelial growth factor has led to the development of anti-angiogenic therapies for the treatment of neoplastic diseases. While not being bound by any theory, the important role of angiogenesis in the pathogenesis of joint disorders such as rheumatodial arthritis led to the suggestion that anti-angiogenic therapy may be a useful adjunct to existing approaches in the treatment of rheumatodial arthritis. See Ballara S. C. et.al., Int J Exp Pathol., 1999, October;80(5):235-50.

It is therefore desirable to provide improved methods of reducing joint related pain that is both less costly and less surgically extreme.

### SUMMARY OF INVENTION

The present invention fills the foregoing need by providing methods for treating joint pain by providing an effective amount of an anti-angiogenic agent to a synovial capsule adjacent to a joint. In particular the anti-angiogenic agent prevents the formation of capillaries within the synovial capsule. While not being bound by any theory applicants believe that prevention of vascularization reduces the inflammation of the joint.

In a further aspect of the invention, the anti-angiogenic agent can be incorporated into a carrier.

In an even further aspect, the anti-angiogenic agent and optionally an additive may be presented in sustained-release formulations.

In an another aspect, the anti-angiogenic agent is adapted to disrupt vascular elements in the synovial capsule region. The active ingredient within the anti-angiogenic agent may include, for example a cell membrane-permeabilizing agent like saporin.

In an another aspect, an anti-neuronal agent is administered with the anti-angiogenic agent to the synovial capsule.

In a further aspect the anti-angiogenic agent can be introduced into the synovial capsule by means of an injection, a pump or a depot.

A preferred embodiment includes administering the anti-angiogenic agent by means of injection into the synovial capsule.

### DETAILED DESCRIPTION OF THE INVENTION

For the purposes of promoting an understanding of the principles of the invention, reference will now be made to preferred embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

Definitions

To aid in the understanding of the invention, the following non-limiting definitions are provided:

The term “angiogenesis” is the name given to the development of new capillaries from pre-existing blood vessels.

The term “anti-angiogenic agent” shall mean any molecule, cell, or physical stimulus which inhibits the growth of blood vessels.

The term “anti-neuronal agent” shall mean any molecule, cell, or physical stimulus which is adapted to inhibit, destroy, force retraction or block further growth of neuronal elements.

The term “additive” shall mean any molecule, cell, intracellular structure, or any combination thereof. By way of non-limiting examples of an “additive”, both a molecule, such as, for example, statin, and a cell, such as, for example, chrondrocyte, are included within the meaning of the term “additive.”

The term “subject” shall mean any animal belonging to phylum Chordata, including, without limitation, humans.

The term “treating” or “treatment” of a disease refers to executing a protocol, which may include administering one or more drugs to a subject (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols which have only a marginal effect on the subject.

The term anti-angiogenic agent and anti-neuronal agent includes one or more of these pharmaceutically active agents unless explicitly stated otherwise.

Anti-angiogenic agents:

Anti-angiogenic agents are any agents that inhibit angiogenesis, whether disclosed herein or known in the art. The extent of angiogenesis is determined by the balance between pro-angiogenic factors and anti-angiogenic factors. Pro-angiogenic factors include, but are not limited to, vascular endothelial growth factor (VEGF), fibroblast growth factor (FGF), interleukin-8 (IL-8), angiogenin, angiotropin, epidermal growth factor (EGF), platelet derived endothelial cell growth factor, transforming growth factor α (TGF-α), transforming growth factor β (TGF-β) , and nitric oxide. Anti-angiogenic factors include, but are not limited to, thrombospondin, angiostatin, and endostatin.

Suitable anti-angiogenic agents include, for example, thalidomide, Neovastat (AE-941) and Rh endostatin.

In a another embodiment, an anti-angiogenic agent is an anti-VEGF agent, such as Macugen™. (Eyetech, New York, N.Y.); or anti-VEGF antibody.

In some embodiments, an anti-angiogenic agent is an antagonist to a VEGF receptor, such as VEGFR1, VEGFR2, VEGFR3, or an antagonist to a VEGF ligand, such as VEGFA, VEGFB, VEGFC, or VEGFD. In some embodiments, an anti-angiogenic agent is antagonist to a VEGF ligand (e.g., VEGFA-VEGFD). Examples of anti-VEGF, anti-angiogenic agents include Avastin (Genentech, Inc.), Macugen (EyeTech Pharmaceuticals, Inc.) or Visudyne (Novartis, Crop.) and anti-VEGF monoclonal antibody M293. Additional examples of anti-VEGF anti-angiogenic agents are disclosed in U.S. Pat. Nos. 5,730,977, 6,383,484, 6,403,088, 6,479,654, 6,559,126, and 6,676,941, all of which are incorporated herein by reference for all intended purposes.

In an embodiment, the anti angiogenic agent may disrupt vascular elements in structures adjacent to the synovial capsule. A cell membrane-permeabilizing agent, for example, saporin is particularly useful in that it can be linked to a molecule that binds to, or is incorporated by, the vascular elements, thus providing for targeted delivery of the saporin to the vascular elements. Molecules to which saporin may be linked include, for example, growth factors, or an antibody or peptide that interacts with the vascular elements. For example, the anti-angiogenic potential of saporin linked to FGF has been explored. See D. A. Lappi et al., Biological and chemical characterization of basic FGF-saporin mitotoxin, 160(2) BIOCHEM. BIOPHYS. RES. COMMUN. 917-23 (1989).

In another embodiment, the anti-angiogenic agent may disrupt the vascular elements by modulating a growth factor or cytokine, or the response of the vascular elements to a growth factor or cytokine; exemplary growth factors or cytokines include vascular growth factor, fibroblast-growth factor, angiopoietins, pigment epithelium-derived factor and α-IFN.

In yet another embodiment, the anti-angiogenic agent may comprise an inhibitory antibody, an aptamer, or a soluble fragment of a growth factor or growth factor receptor; exemplary anti-angiogenic agents include bevacizumab, pegaptanib and ranibizumab. In still another embodiment, the anti-angiogenic agent may comprise an anti-angiogenic steroid or an angiostatic steroid, such as anecortave acetate or triamcinolone acetonide.

In a further embodiment, the anti-angiogenic agent may modulate an extra-cellular matrix component, or the response of the vascular elements to an extra-cellular matrix component; the extra-cellular matrix component may be an MMP inhibitor, such as marimastat; alternatively, the extra-cellular matrix component may be cell adhesion molecules, such as cadherins or integrins. Or, for yet another embodiment, the active ingredient may modulate a cytoskeletal component of established or actively growing vascular extensions, such as microtubules; exemplary active ingredients include combretastatin, vinca alkaloid or placlitaxel.

Anti-Neuronal Agent:

The present invention may further comprise an anti-neuronal agent.

In one embodiment, the anti-neuronal agent is adapted to destroy, force retraction or block the further growth of the neuronal elements in the synovial capsule or synovial capsule region. In a particular embodiment, the anti-neuronal agent comprises a neurotoxin and cell membrane-permeabilizing agents, which may be, for example, a natural compound, a synthetic compound, a dye, saponins or saporin.

In yet another embodiment, the anti-neuronal agent is adapted to modulate an extra-cellular matrix component, or to modulate the response of the neuronal elements to an extra-cellular matrix component. Exemplary extra-cellular matrix components include chondroitin sulfate proteglycans, netrins, semaphorins and myelin/oligodendrocyte growth inhibitors, such as Nogo, MAG and Omgp. Alternatively, the extra-cellular matrix component may be a cell adhesion molecule, such as NCAM, N-cadherins and integrins. As an exemplary active ingredient, Semaphorin III protein can repulse sensory neuronal extensions that are invading a site of injury and inflammation. See D. L. Tanelian et al., Semaphorin III can repulse and inhibit adult sensory afferents in vivo, 3(12) NAT. MED. 1398-401 (1997). Function-blocking anti-integrin antibodies that can reduce the development of hyperalgesia may also serve as suitable active ingredients. See O. A. Dina et al., Primary afferent second messenger cascades interact with specific integrin subunits in producing inflammatory hyperalgesia, 115(1-2) PAIN 191-203 (2005).

In another embodiment, the anti-neuronal agent is adapted to modulate a cytoskeletal component, or the organization of established or actively growing neuronal elements. Exemplary cytoskeletal components may include actins, tubulins or neurofilaments, and an exemplary active ingredient for the anti-neuronal agent may be a Rho kinase activator. Cytotoxic necrotizing factor-1 and -2 from E.coli may be suitable Rho kinase activators.

In one embodiment, the anti-neuronal agent is adapted to desensitize the neuronal elements in the disc or disc region. Various specific embodiments include selecting an active ingredient for the anti-neuronal agent, such as camphor, menthol, piperine, mustard oil, curcumin and eugenol. Other embodiments include the use of a vanilloid receptor agonist for the active ingredient, such as 8-Methyl-N-vanillyl-trans-6-nonenamide (Capsaicin); Z-Capsaicin; Gingerol; Zingerone; 8-Methyl-N-vanillylnonanamide (Dihydrocapsaicin); 6,7-Deepoxy-6,7-didehydro-5-deoxy-21-dephenyl-21-(phenylmethyl)-daphnetoxin, 20-(4-hydroxy-5-iodo-3-methoxybenzeneacetate) (5′-Iodoresiniferatoxin)-?; (+)-Isovelleral; N-Vannilyloleoylamide (Olvanil); Phorbol 12,13-dinonanoate 20-homovanillate; Resiniferatoxin; N-(3-Methoxyphenyl)-4-chlorocinnamide (SB-366791); 2,3,4-Trihydroxy-6-methyl-5-[(2E,6E)-3,7,11-trimethyl-2,6,10-dodecatrienyl]benzaldehyde (Scutigeral); 6,7-Deepoxy-6,7-didehydro-5-deoxy-21-dephenyl-21-(phenylmethyl)-20-(4-hydroxybenzeneacetate)daphnetoxin (Tinyatoxin); capsaicin synthetics; and capsaicin derivatives, and any combination thereof.

In another embodiment, the anti-neuronal agent is adapted to modulate the neuronal elements in the synovial capsule. In a specific embodiment, the anti-neuronal agent modulates the activity of the neuronal elements innervating the synovial capsule or synovial capsule region. For example, the anti-neuronal agent may modulate neuronal receptors, such as nociceptors, vanilloid, adrenergic, cholinergic, glutamate, GABA, serotonine, somatostatin opioids, ATP, Na+, K+, Ca2+, cannabinoids, Substance P and neuropeptide receptors; suitable active ingredients may include botulinum toxin, anti-convulsants, anesthetics, analgesics, opioids and cannabinoids. Other suitable active ingredients include vanilloid receptor antagonists, such as N-[2-(4-Chlorophenyl)ethyl]-1,3,4,5-tetrahydro-7,8-dihydroxy-2H-2-benzazepine-2-carbothioamide (Capsazepine); [N-(4-Hydroxy-3-methoxyphenyl)methyl]-5Z,8Z,11Z,14Z-eicosatetraenamide](Arvanil); N-(3-Methoxyphenyl)-4-chlorocinnamide (SB-366791) and 5′-iodoresiniferatoxin.

In another embodiment, the anti-neuronal agent may cause reduction in the sensitization of the neuronal elements by a pro-inflammatory molecule. Exemplary pro-inflammatory molecules include cytokines, chemokines, neuropeptides, bradykinin, histamine and prostaglandins. Suitable active ingredients may include steroids, nonsteroidal anti-inflammatory drugs, Cox inhibitors, modulators of TNF-alpha or IL-1 cytokine levels or receptors, or NFkB modulators, or any combination thereof.

In yet another embodiment, the anti-neuronal agent is adapted to modulate glial cells. In specific embodiments, the anti-neuronal agent comprises fluorocitrate or minocyclin.

The term “neuronal element” includes a neuron body; extensions of a neuron, such as axons, axonal branches, dendrites or growth cones; and supporting cells, such as glial cells including astrocytes, Schwann cells and microglia. The one or more active ingredients in the anti-neuronal agent may achieve disruption of neuronal elements by reducing or suppressing activation of the neuronal elements, preventing or reducing the formation of such neuronal elements in the synovial capsule region, or destroying, inducing retraction or modulating further growth of the neuronal elements already present in the synovial capsule region.

In one embodiment, the anti-neuronal agent may modulate the neuronal elements in the synovial capsule region. More specifically, the anti-neuronal agent may modulate the activity of the neuronal elements in the synovial capsule region. In one variation, the anti-neuronal agent modulates the sensitivity of neuronal receptors, such as nociceptors, vanilloid, adrenergic, cholinergic, glutamate, GABA, serotonine, somatostatin opioids, ATP, Na+, K+, Ca2+, cannabinoids, Substance P and neuropeptide receptors. Suitable active ingredients in this variation includes botulinum toxin, anti-convulsants, anesthetics, analgesics, opioids and cannabinoids. In another variation, a vanilloid receptor antagonist is used for the active ingredient; exemplary compounds for the active ingredient include N-[2-(4-Chlorophenyl)ethyl]-1,3,4,5-tetrahydro-7,8-dihydroxy-2H-2-benzazepine-2-carbothioamide (Capsazepine); [N-(4-Hydroxy-3-methoxyphenyl)methyl]-5Z,8Z,11Z,14Z-eicosatetraenamide] (Arvanil); N-(3-Methoxyphenyl)-4-chlorocinnamide (SB-366791) and 5′-iodoresiniferatoxin, and any combination thereof.

Carriers:

The anti-angiogenic agent compound may be included into a carrier which is administered into the synovial capsule. Suitable non-limiting examples of the carriers include a gel, such as, for example, a PEG gel. The methods of incorporating the anti-angiogenic agent into the carrier are known to a person of ordinary skill in the art and depend on the nature of the anti-angiogenic agent and the nature of the carrier selected by a person practicing the current invention. Ionic binding, gel encapsulation or physical trapping inside the carrier, iontophoresis and soaking the carrier in a solution of the anti-angiogenic agent are suitable examples of such methods.

Additives:

In different embodiments of the invention, an additive may also be added to the carrier. The additive may include a growth factor, an antibiotic, an analgesic, a cell, and any combination thereof.

Suitable growth factors include, without limitation, BMP-2, rhBMP-2, BMP-4, rhBMP-4, BMP-6, rhBMP-6, BMP-7[OP-1], rhBMP-7, GDF-5, LIM mineralization protein, platelet derived growth factor (PDGF), transforming growth factor β (TGF-β), insulin-related growth factor-I (IGF-I), insulin-related growth factor-II (IGF-II), fibroblast growth factor (FGF), beta-2-microglobulin (BDGF II), rhGDF-5, and any combination thereof.

Suitable cell teipes include, without limitation, mesenchymal stem cells, periosteal cells, pluripotent stem cells, embryonic stem cells, osteoprogentior cells, osteoblasts, osteoclasts, bone marrow-derived cell lines, and any combination thereof.

Suitable analgesics include morphine and naloxone), local anaesthetics (such as, for example, lidocaine), glutamate receptor antagonists, □-adrenoreceptor agonists, adenosine, canabinoids, cholinergic and GABA receptors agonists, and different neuropeptides. A detailed discussion of different analgesics is provided in Sawynok et al., (2003) Pharmacological Reviews, 55:1-20, the content of which is incorporated herein by reference.

Suitable antibiotics include, without limitation nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol and any combination thereof.

A person of ordinary skill in the art will appreciate that the anti-angiogenic agent and the anti-neuronal agent may also be delivered on the carrier and/or in a sustained-release formulation.

Sustained-Release Formulations:

In another embodiment of the present invention, the anti-angiogenic agent, and, optionally, the additive may be presented in a sustained-release formulation. Suitable sustained-release formulations include but not limited to capsules, microspheres, particles, gels, coating, matrices, wafers, pills or other pharmaceutical delivery compositions. The examples of such sustained-release formulations have been described previously, for example, in U.S. Pat. Nos. 6,953,593, 6,946,146, 6,656,508, 6,541,033, 6,451,346, the contents of which are incorporated herein by reference. Many methods of preparation of a sustained-release formulation are known in the art and are disclosed in Remington's Pharmaceutical Sciences (18th ed.; Mack Publishing Company, Eaton, Pa., 1990), incorporated herein by reference.

Generally, the anti-angiogenic agent can be entrapped in semipermeable matrices of solid hydrophobic polymers. The matrices can be shaped into films or microcapsules. Examples of such matrices include, but are not limited to, polyesters, copolymers of L-glutamic acid and gamma ethyl-L-glutamate (Sidman et al. (1983) Biopolymers 22:547-556), polylactides (U.S. Pat. No. 3,773,919 and EP 58,481), polylactate polyglycolate (PLGA) such as polylactide-co-glycolide (see, for example, U.S. Pat. Nos. 4,767,628 and 5,654,008), hydrogels (see, for example, Langer et al. (1981) J. Biomed. Mater. Res. 15:167-277; Langer (1982) Chem. Tech. 12:98-105), non-degradable ethylene-vinyl acetate (e.g. ethylene vinyl acetate disks and poly(ethylene-co-vinyl acetate)), degradable lactic acid-glycolic acid copolyers such as the Lupron Depot™, poly-D-(−)-3-hydroxybutyric acid (EP 133,988), hyaluronic acid gels (see, for example, U.S. Pat. No. 4,636,524), alginic acid suspensions, polyorthoesters (POE), and the like.

Suitable microcapsules can also include hydroxymethylcellulose or gelatin-microcapsules and polymethyl methacrylate microcapsules prepared by coacervation techniques or by interfacial polymerization. See the PCT publication WO 99/24061 entitled “Method for Producing Sustained-release Formulations,” wherein a protein is encapsulated in PLGA microspheres, herein incorporated by reference. In addition, microemulsions or colloidal drug delivery systems such as liposomes and albumin microspheres, may also be used. See Remington's Pharmaceutical Sciences (18^{th} ed.; Mack Publishing Company Co., Eaton, Pa., 1990). Other preferred sustained-release compositions employ a bioadhesive to retain anti-angioaenic agent at the site of administration.

The sustained-release formulation may comprise a biodegradable polymer, which may provide for non-immediate release. Non-limiting examples of biodegradable polymers suitable for the sustained-release formulations include poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, polyorthoesters (POE), or any combinations thereof, as described, for example, in the U.S. Pat. No. 6,991,654 and U.S. Pat. Appl. No. 20050187631, each of which is incorporated herein by reference in its entirety.

A person of ordinary skill will appreciate that different combinations of the sustained-release formulations are also suitable for this invention. For example, the practitioner may formulate the at least one anti-inflammatory compound as a combination of a gel and microspheres loaded with the at least one anti-inflammatory compound, wherein the combination of gel and microspheres are placed in the bone defect.

In the practice of the invention, the administration is localized and sustained. For example, depending on the carrier, the sustained-release formulations, and the total amount of the anti-angiogenic agent, the anti-neuronal agent, the practitioner can choose a combination, which will release the active material over a desired time period ranging between about one day and about six months.

In yet other embodiments, further excipients are employed. The amount of excipient that is useful in the composition of this invention is an amount that serves to uniformly distribute the anti-angiogenic agent throughout the composition so that it can be uniformly dispersed when it is to be delivered to a subject in need thereof. It may serve to dilute the anti-angiogenic agent to a concentration at which the anti-angiogenic agent can provide the desired beneficial palliative or curative results while at the same time minimizing any adverse side effects that might occur from too high a concentration. It may also have a preservative effect. Thus, for the anti-angiogenic agent that has high physiological activity, more of the excipient will be employed. On the other hand, for the anti-angiogenic agent compound that exhibits a lower physiological activity a lesser quantity of the excipient will be employed. In general, the amount of excipient in the composition will be between about 50% weight (w) and 99.9% w. of the total composition. For the anti-angiogenic agent that has a particularly high physiological activity, the amount will be between about 98.0% and about 99.9% w.

Accordingly, the methods of creating the sustained-release formulations comprising the at least one anti-inflammatory compounds and/or the additive are within the expertise of the person having ordinary skill in the art.

The anti-angiogenic agent may be administered locally. In one embodiment, anti-angiogenic agent has a targeted release rate, and is injected into the synovial capsule. In another embodiment, a controlled administration system releases the anti-angiogenic agent. The controlled administration system may be, for example, a depot, an infusion pump, an osmotic pump, or an interbody pump. The controlled administration system may be implanted adjacent to the synovial capsule.

In yet another embodiment, the controlled administration system comprises a system administered locally by insertion of a catheter at or near a target site, the catheter having a proximal end and a distal end, the proximal end having an opening to deliver a pharmaceutical in situ, the distal end being fluidly connected to a pharmaceutical delivery pump. For example, the proximal end of the catheter delivers the anti-angiogenic agent within 10 cm of the synovial capsule, more particularly, within 5 cm of the target site.

A depot includes but is not limited to capsules, microspheres, particles, gels, coatings, matrices, wafers, pills or other pharmaceutical delivery compositions for containing one or more active ingredients. A depot may comprise a biopolymer. The biopolymer may provide for non-immediate release of the one or more active ingredients. Examples of suitable sustained release biopolymers include but are not limited to poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, and combinations thereof.

A controlled administration system provides localized delivery of one or more active ingredients in a quantity of therapeutic agent that can be deposited at the target site as needed for pain, either continuously or at an intermittent rate. A controlled administration system includes, but is not limited to, a bolus of the therapeutic agent, a depot, an osmotic pump, an interbody pump, an infusion pump, implantable mini-pumps, a peristaltic pump, other pharmaceutical pumps, or a system administered locally by insertion of a catheter into, at or near the synovial capsule, the catheter being operably connected to a pharmaceutical delivery pump. It is understood that pumps can be internal or external as appropriate.

The anti-angiogenic agent and/or the anti-neuronal agent, and optionally, the additive may be injected into the synovial capsule. This embodiment is especially preferable. Specific embodiments of administering the anti-angiogenic agent can be found at Trieu et.al., U.S. Pat. Appl. No. 2004005414, U.S. Pat. Appl. No. 20040228901, U.S. Pat. Appl. No. 200540119754, and U.S. Pat. Appl. No. 20050197707.

A person skilled in the art will appreciate that various modifications of this embodiment are possible. Among these modifications are different sustained-release formulations of the anti-angiogenic agent and/or the anti-neuronal agent and additive.

The techniques of pain assessment include, without limitation, VAS, Oswestri, and SF-36 Questionnaires. Alternatively, the pain may be assessed based on bone hydration level, type II collagen level, proteoglycan levels, and any combination of the techniques disclosed above.

Specific embodiments according to the methods of the present invention will now be described in the following non-limiting examples. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

### EXAMPLE

### Example 1

An anti-angiogenic agent may be mixed with biocompatible medium such as water, saline, or ethylene glycol and injected directly into the synovial capsule using a syringe and a hypodermic needle. The anti-angiogenic agent is contained within the joint capsule following injection. A single injection is effective for reducing the pain, although additional injections may be necessary to achieve appropriate level of treatment.

### Example 2

An anti-angiogenic agent and an anti-neuronal agent may be mixed with biocompatible medium such as water, saline, or ethylene glycol and injected directly into the synovial capsule using a syringe and a hypodermic needle. The anti-angiogenic agent and the anti-neuronal agent is contained within the joint capsule following injection. A single injection is effective for reducing the pain, although additional injections may be necessary to achieve appropriate level of treatment.

### Example 3

A gel or suspension of hydrated carrier having particulates and/or fibrous material is soaked with the anti-angiogenic agent in a biocompatible medium such as water, saline or ethylene glycol. The suspension is injected directly into the synovial capsule using a hypodermic needle. The suspension containing the anti-angiogenic agent is contained within the synovial capsule following injection. The medium subsequently diffuses out of the disc space and leaves the carrier and the anti-angiogenic agent behind. Single injection is desirable; however, additional injections may be necessary to achieve the appropriate level of treatment.

### Example 4

A gel or suspension of hydrated carrier having particulates and/or fibrous material is soaked with the anti-angiogenic agent and the anti-neuronal agent in a biocompatible medium such as water, saline or ethylene glycol. The suspension is injected directly into the synovial capsule using a hypodermic needle. The suspension containing the anti-angiogenic agent is contained within the synovial capsule following Injection. The medium subsequently diffuses out of the disc space and leaves the carrier and the anti-angiogenic agent behind. Single injection is desirable; however, additional injections may be necessary to achieve the appropriate level of treatment.

### Example 5

Dehydrated carrier material in granule, particulate or powder form containing the anti-angiogenic agent loaded into a specially designed syringe for delivery of particulate matter. Such a syringe is described in the embodiments of U.S. Pat. Appl. No.20040228901 to Trieu et. al. The material is extruded into the synovial capsule through a small, dilated opening. The material remains inside the synovial capsule after the needle is removed. The material subsequently absorbs moisture or body fluids and swells up in vivo. Single injection is desirable; however, additional injections may be necessary to achieve the appropriate level of treatment.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

We claim:

1. A method for treating a joint pain in a subject comprising providing an effective amount of an anti-angiogenic agent to a synovial capsule adjacent to the joint.

2. The method of claim 1, wherein the anti-angiogenic agent is incorporated into a carrier.

3. The method of claim 1, wherein the anti-angiogenic agent is selected from the group consisting of thalidomide, Neovastat (AE-941),Rh endostatin and anti-VEGF agents.

4. The method of claim 4, wherein the anti-angiogenic agent is thalidomide.

5. The method of claim 1, wherein the anti-angiogenic agent is provided in a sustained-release formulation.

6. The method of claim 1, further comprising providing an effective amount of an anti-neuronal agent to the synovial capsule adjacent to the joint.

7. The method of claim 6, wherein the anti-neuronal agent is incorporated into a carrier.

8. The method of claim 6, wherein the anti-neuronal agent is selected from the group consisting of nociceptors, vanilloid, adrenergic, cholinergic, glutamate, GABA, serotonine, somatostatin opioids, ATP, Na+, K+, Ca2+, cannabinoids, Substance P and neuropeptide receptors, and any combination thereof.

9. The method of claim 6, wherein the anti-neuronal agent is selected from group consisting of N-[2-(4-Chlorophenyl)ethyl]-1,3,4,5-tetrahydro-7,8-dihydroxy-2H-2-benzazepine-2-carbothioamide (Capsazepine); [N-(4-Hydroxy-3-methoxyphenyl)methyl]-5Z,8Z,11Z,14Z-eicosatetraenamide] (Arvanil); N-(3-Methoxyphenyl)-4-chlorocinnamide (SB-366791) and 5′-iodoresiniferatoxin, and any combination thereof.

10. The method of claim 1, further comprising providing an additive to the synovial capsule adjacent to the joint, the additive selected from the group consisting of growth factors, cells, antibiotics, analgesics, and any combination thereof.

11. The method of claim 10, wherein the additive is incorporated into a carrier.

12. The method of claim 10, wherein the additive is a growth factor selected from the group consisting of BMP-2, rhBMP-2, BMP-4, rhBMP-4, BMP-6, rhBMP-6, BMP-7[OP-1], rhBMP-7, GDF-5, LIM mineralization protein, platelet derived growth factor (PDGF), transforming growth factor β (TGF-β), insulin-related growth factor-I (IGF-I), insulin-related growth factor-II (IGF-II), fibroblast growth factor (FGF), beta-2-microglobulin (BDGF II), rhGDF-5, and any combination thereof.

13. The method of claim 12, wherein the growth factor is BMP-2.

14. The method of claim 10, wherein the additive is a cell selected from the group consisting of a mesenchymal stem cell, periosteal cell, pluripotent stem cell, embryonic stem cell, osteoprogentior cell, osteoblast, osteoclast, bone marrow-derived cell line, and any combination thereof.

15. The method of claim 1 wherein the joint is selected from the group consisting of knee joints, hip joints, ankle joints, finger joints and shoulder joints.

16. The method of claim 1 wherein the Joint is a knee joint.

17. The method of claim 1, wherein the anti-angiogenic agent is administered through an injection, pump, and depot.

18. The method of claim 1, wherein the anti-angiogenic agent is administered through an injection.

19. The method of claim 1, wherein the anti-angiogenic agent prevents and/or inhibits the vacularization of the synovial capsule.

20. A method for treating a knee joint pain in a subject comprising providing an effective amount of an anti-angiogenic agent to a synovial capsule adjacent to the knee joint wherein the anti-angiogenic agent is administered through an injection, pump, and depot.
